SEQUENCE LISTING (2 sequences)

SEQ ID NO: 1 - DNA, 45 bp, Artificial Sequence
  Synthetic
aatttaatacgactcactatagggaagtagttatccccctccatc

SEQ ID NO: 2 - DNA, 21 bp, Artificial Sequence
  Synthetic
gatacggattcttaacgtcgc

(19) United States
(12) Patent Application Publication
(10) Pub. No.: US 2013/0302814 A1
(43) Pub. Date: Nov. 14, 2013

(54) PROCESS FOR ISOLATING MICROORGANISMS

(71) Applicant: Blood Cell Storage, Inc., Seattle, WA (US)

(72) Inventor: Paul V. Haydock, Shoreline, WA (US)

(21) Appl. No.: 13/908,810

(22) Filed: Jun. 3, 2013

Related U.S. Application Data
(63) Continuation of application No. PCT/US2011/063337, filed on Dec. 5, 2011.
(60) Provisional application No. 61/419,669, filed on Dec. 3, 2010.

(51) Int. Cl. C12Q 1/24; C12Q 1/68

(52) U.S. Cl. CPC C12Q 1/24; C12Q 1/6806
USPC 435/6.12; 435/30; 435/6.15

(57) ABSTRACT

A process for isolating microorganisms is disclosed. The process utilizes a device comprising an inner surface, an outer surface, a first port, and a second port, wherein the inner surface comprises an unmodified, smooth glass substrate and defines a binding chamber providing fluid communication between the first port and second port. Microorganisms in an aqueous solution are contacted with the unmodified, smooth glass substrate, wherein the solution is essentially free of cell precipitants, and the microorganisms are allowed to bind to the glass substrate.

### CROSS-REFERENCE TO RELATED APPLICATION

This application is a continuation of International Application No. PCT/US2011/063337, filed Dec. 5, 2011, which claims the benefit of U.S. Provisional Application No. 61/419,669, filed Dec. 3, 2010, each of which is incorporated herein by reference in its entirety.

### BACKGROUND OF THE INVENTION

Isolation of microorganisms and analysis of their component nucleic acids has a wide range of applications in medicine, public health, industry, forensics, and research. Microorganisms of interest may be present in small amounts, and their isolation may require filtration and concentration of large volumes of samples. Many nucleic acid-based assays require substantial amounts of often rare nucleic acids. It has proven difficult and/or costly to extract sufficient quantities of some rare nucleic acid species using conventional methods. For example, environmental monitoring, including testing of water samples for viral or other pathogen contamination, requires processing of large volumes of sample due to the often dilute nature of the contamination. Such processing commonly involves concentration by filtration and requires multiple steps to reduce input volumes to the microliter to milliliter size used in many assays. Dilute nucleic acid samples are difficult to extract with conventional methods such as spin silica columns, magnetic beads, or solution-based methods such as the phenol-chloroform extraction process. The silica particle methods use low volume vessels and require multiple loading and tedious manual methods. The solution-phase methods can use large vessels, but for dilute samples there is no visible nucleic acid, and failures are frequent in the precipitation step.

Rapid analysis of nucleic acids from biological samples has been advanced by the development of microfluidic technologies capable of extracting nucleic acids from cell lysates and other sources. Rapid extraction methodologies can be combined with amplification techniques such as polymerase chain reaction (PCR) to provide useful quantities of nucleic acids from minute samples of blood, tissue, cultured cells, or other biological materials. These microfluidic technologies have been widely adopted in biomedical research laboratories, permitting, for example, high-throughput screening of cloned DNA “libraries” from cultured bacteria or other host cells. However, conventional methods of isolating microorganisms and extracting and analyzing their nucleic acids still require extensive handling and processing of samples.

There remains a need in the art for efficient methods of capturing microorganisms and their component nucleic acids from biological samples, including high-volume, dilute samples. There remains a further need for such capture methods that do not rely on specific cell-binding or nucleic acid-binding agents, such as antibodies, ligands, oligonucleotides, or the like. The present invention provides such methods, as well as other, related advantages.

### SUMMARY OF THE INVENTION

The present invention provides processes for isolating microorganisms. The processes comprise the steps of (a) providing a device comprising an inner surface, an outer surface, a first port, and a second port, wherein the inner surface comprises an unmodified, smooth glass substrate and defines a binding chamber providing fluid communication between the first port and the second port; (b) contacting microorganisms in an aqueous solution with the unmodified, smooth glass substrate, wherein the solution is essentially free of cell precipitants; and (c) allowing the microorganisms to bind to the glass substrate to provide bound cells.

Within one embodiment, the process further comprises separating the aqueous solution from the bound microorganisms. Within a related embodiment the process further comprises, following the separating step, adding a growth medium to the bound microorganisms and incubating the bound microorganisms, whereby the bound microorganisms increase in number.

Within another embodiment, the process further comprises lysing the bound microorganisms to produce a lysate. Within a related embodiment, a chaotropic salt is added to the lysate, whereby nucleic acid within the lysate binds to the unmodified, smooth glass surface, and the bound nucleic acid is washed to provide isolated nucleic acid. Within another related embodiment, the isolated nucleic acid is amplified, such as by isothermal amplification. Within one embodiment, the isolated nucleic acid is amplified with the binding chamber.

Within a further embodiment, the microorganisms are single-cell microorganisms. Within related embodiments, the microorganisms are bacteria, or the microorganisms are Gram-negative bacteria. Within another embodiment, the microorganisms are yeast.

Within an additional embodiment, the aqueous solution is essentially free of mineral salts.

Within another embodiment, the aqueous solution comprises a detergent.

Within a further embodiment, the aqueous solution is a low-ionic-strength solution. Within a related embodiment, the aqueous solution has an ionic strength less than 0.1 M.

Within another embodiment, the aqueous solution is free of aliphatic alcohols.

Within another embodiment, the aqueous solution comprises blood or a blood component.

Within another embodiment, the unmodified, smooth glass substrate is flat. Within a related embodiment, the binding chamber is a serpentine chamber of rectangular cross-section. Within another related embodiment, the serpentine chamber is planar.

Within a further embodiment, the binding chamber encloses a volume x, and a volume of the aqueous solution of at least 2× is contacted with the glass substrate. Within a related embodiment, a volume of the aqueous solution of at least 10× is contacted with the glass substrate.

These and other aspects of the invention will become evident upon reference to the following detailed description of the invention and the attached drawings.

All references cited herein are incorporated by reference in their entirety. Numeric ranges recited herein include the endpoints.

### BRIEF DESCRIPTION OF THE FIGURES

FIG. 1 illustrates a representative extraction vessel that can be used within the present invention.

FIG. 2 shows the results of an experiment in which bacteria were captured in a glass-walled device in the presence and absence of magnetic beads and antibodies. M, molecular weight markers. (−), NASBA negative control. (+), NASBA positive control. 1, magnetic beads only. 2, magnetic beads with bacteria. 3, antibody with bacteria. 4, magnetic beads with antibody and bacteria. 5, beads, antibody, and blood. 6, beads, bacteria, antibody, and blood.

FIG. 3 shows the results of an experiment in which bacteria were captured in a glass-walled device in the presence and absence of magnetic beads and antibodies. M, molecular weight markers. 1, beads only. 2, antibody only. 3, bacteria only. 4, antibody and bacteria. 5, beads with antibody. 6, beads, antibody, and bacteria. 7, beads, antibody, and blood. 8, beads, antibody, blood, and bacteria.

FIG. 4 illustrates the results of an experiment in which bacteria were captured on unmodified, smooth glass at various pHs in the presence or absence of mannose. (−), NASBA negative control; (+), NASBA positive control; C, Control sample in which bacteria are suspended in water alone (no pH adjustment, no albumin blocker); First Elution, 75-μl elution over dry channel; Second Elution, 75-μl elution over previously eluted channel (to recover residual nucleic acid).

FIG. 5 illustrates the effect of increasing the volume of bacteria in a sample on nucleic acid yield.

FIG. 6 illustrates the effect of bacterial suspension dilution on nucleic acid yield.

FIG. 7 illustrates the viability of bacterial cells bound to glass. Lanes are: M, molecular weight markers; 1, NASBA negative control; 2, NASBA positive control; S, cells bound to glass and starved in water; F, starved cells bound to glass, revived while still bound to glass in broth.

FIG. 8 illustrates the effects of antibiotics on cultured, glass-bound bacteria. Lanes are: 1, NASBA negative control; 2, NASBA positive control; 3, starved cells; 4, starved, then fed; 5, starved, fed with 50 μg/ml Ampicillin; 6, same as 5, then fed without Ampicillin; 7, starved, fed with 50 μg/ml Tetracycline; 8, same as 7, then fed without Tetracycline.

FIG. 9 illustrates the results of an experiment in which cells were bound to a glass substrate in the presence of detergent.

### DETAILED DESCRIPTION OF THE INVENTION

The present invention provides methods for isolating microorganisms, including single-cell microorganisms, by binding them to glass. Of particular interest are bacteria and yeasts. Within certain embodiments, the isolated microorganisms are cultured on the glass to increase their numbers, facilitating the isolation and analysis of microorganisms that occur in very low concentrations. In other embodiments, the microorganisms are lysed to release their nucleic acids. The nucleic acids are then captured by binding them to the glass, and can be analyzed, quantitated, and/or amplified within the isolation device. The invention thus provides a system for isolating, identifying, quantitating, and analyzing microorganisms and their component nucleic acids. This technology has a wide range of applications, including medical diagnostics, sterility testing, industrial QA/QC, and environmental monitoring.

Unlike known methods that capture bacteria and other microorganisms using specific binding moieties such as antibodies, the methods of the invention utilize unmodified, smooth glass substrates that bind the microorganisms non-specifically. There are many high-value instances where the use of a specific binding moiety is not preferred. One such instance is the diagnosis of blood infections. There should be no bacteria present in blood, yet there are a number of bacteria that can cause infections. To use a technology that relies on specific binding agents, one would need a dozen or so binding moieties, which presents a significant technical challenge and would be expensive to the end-user. The use of unmodified, smooth glass in the present invention allows the capture of all bacteria in their correct proportions on a clean glass surface so that after collecting nucleic acids, the various species present can be identified using well-established techniques, such as amplification by polymerase chain reaction (PCR; see, e.g., Mullis, U.S. Pat. No. 4,683,202) or nucleic acid sequence-based amplification (NASBA; Malek et al., U.S. Pat. No. 5,130,238).

The invention provides a number of other advantages. First, it is simple to use and can therefore be implemented in point-of-care diagnosis and in remote locations. Second, it can be used with existing devices, such as Pasteur pipettes and S-channel nucleic acid extraction cards (e.g., as disclosed by Reed et al., U.S. Pat. No. 7,608,399 and Reed et al., U.S. Patent Application Publication No. 20090215125 A1). Such devices provide a smooth capture surface that facilitates removal of contaminants by washing. Third, microorganisms bound to glass have been found to remain viable under the capture conditions used within the invention. Fourth, glass binds many species of bacteria and yeast. Fifth, the methods can be used to isolate microorganisms from complex biological samples, including blood and blood products. Sixth, the methods are conveniently coupled with downstream nucleic acid isolation and analysis within a single device.

In addition, the methods of the invention provide for the isolation of microorganisms from dilute samples without the need for centrifugation or filtration. Centrifugation is generally inconvenient, and losses of microorganisms (especially from dilute suspensions) can occur. Although filtration methods are known to be useful for some sample types, other substances or other types of cells can also be captured on the filters and may interfere with subsequent analysis. In some instances, filters may not be the ideal surface for further analysis. In contrast, glass is relatively inert and can be used (and has been used) in a variety of assay formats.

The term “essentially free” is used herein to denote the absence of functionally significant quantities of a component; however, detectable trace quantities may be present. For example, a solution that is essentially free of cell precipitants is free of such components in amounts sufficient to effect measurable precipitation of cells.

As used herein, a “low ionic strength” solution is a solution having an ionic strength less than 300 mM. Within certain embodiments of the invention, the ionic strength of such a solution is less than 200 mM, less than 100 mM, less than 75 mM, less than 50 mM, or less than 20 mM.

“Nucleic acid” includes deoxyribonucleic acid (DNA), ribonucleic acid (RNA), and mixtures thereof, including naturally occurring and synthesized forms. “Nucleic acid” includes chromosomal and extrachromosomal forms, and fragments thereof. The singular form of the term includes mixtures of molecules, including molecules of varying size, source, and chemical composition (including mixtures of DNA and RNA).

“Viable” microorganisms are microorganisms that are able to multiply when provided with the requisite nutrients and environmental conditions, such as temperature. As will be recognized by those of ordinary skill in the art, nutritional and other environmental requirements vary among species. Determination of growth requirements for a particular species is within the level of ordinary skill in the art.

As used herein, an “unmodified smooth glass substrate” means a glass substrate having a surface smoothness corresponding to that of a standard microscope slide, Pasteur pipette, glass capillary, or the like, wherein the substrate has not been etched or otherwise altered to increase its surface area, and wherein it has not been modified to specifically bind nucleic acids or cells as disclosed below. Specifically excluded from “smooth glass” is porous glass that is known in the art to capture nucleic acids, commonly in bead, frit, or membrane form. Such porous glass commonly has pores sized within the range of 0.1 μm to 300 μm. Suitable glass materials for use within the present invention include, without limitation, soda lime glass (e.g., Erie Electroverre Glass; Erie Scientific Company, Portsmouth, N.H.), borosilicate glass (e.g., Corning 0211, PYREX 7740; Corning Incorporated, Corning, N.Y.), zinc titania glass (Corning Incorporated), and silica glass (e.g., VYCOR 7913; Corning Incorporated). The glass substrate is essentially free of nucleic acid-specific binding sites, such as charged surfaces or binding sites provided by immobilized oligonucleotides, minor groove binding agents, intercalating agents, or the like. A substrate that is “essentially free of nucleic acid-specific binding sites” is one that does not contain an amount of such sites sufficient to give a statistically significant increase in nucleic acid binding as compared to glass. The glass substrate is also essentially free of cell-specific binding sites, such as antibodies, immobilized receptors, immobilized ligands, and the like, i.e., it does not contain an amount of such sites sufficient to give a statistically significant increase in cell binding as compared to glass.

The present invention provides methods wherein microorganisms are isolated by binding to unmodified, smooth glass substrates. These substrates are also effective for binding nucleic acids, including DNA, RNA, and mixtures thereof. Thus, within certain embodiments the invention provides methods wherein microorganisms are bound to a glass substrate, lysed, and the released nucleic acids are captured by binding to the same glass substrate. The glass substrate is conveniently provided within an extraction device comprising an inner surface, an outer surface, a first port, and a second port, wherein the inner surface comprises an unmodified, smooth glass substrate and defines a binding chamber providing fluid communication between the first port and second port. The device is configured to permit fluid flow through the chamber and across the smooth glass substrate(s), with the ports providing fluid communication between the chamber and the external environment. The first and second ports thus provide for introduction and removal of samples, reagents, and gasses. Such vessels include, for example, laminated devices comprising glass-walled, serpentine channels (“S-channels”) as disclosed by Reed et al., U.S. Pat. No. 7,608,399 and Reed et al., U.S. Patent Application Publication No. 20090215125 A1. Other suitable vessels include Pasteur pipettes, other types of glass tubing, coaxial assemblies of glass tubing and/or rods, glass capillaries, chromatography columns, condenser tubes, syringes, and the like as disclosed by Reed et al., U.S. Patent Application Publication No. 20110203688 A1. Vessels of this type can extract microorganisms from large-volume samples, which can be flowed through the chamber via the first and second ports at a rate selected to provide the desired contact time between the sample and the glass. In an alternative mode of operation, large samples can be divided into aliquots of a volume less than or equal to the volume of the chamber and processed in batch mode. The invention eliminates the need for a separate filtration (concentration) step in order to capture microorganisms from many dilute samples, although a concentrating step may be added to reduce sample volume if desired.

Extraction devices used within the present invention will ordinarily have binding chamber volumes in the microliter to milliliter range, commonly 200 μL to 20 mL, more commonly 500 μL to 10 mL, and often 0.75 mL to 2 mL. In some instances a chamber size larger than the selected sample volume will be used in order to allow the sample to be exposed to a larger substrate surface area.

The extraction devices of Reed et al. (ibid.) include simple, microtiter plate-sized nucleic acid extraction devices wherein glass microscope slides provide the unmodified, smooth glass substrate. This type of device (commonly referred to as a “card” or “S-channel card”) works especially well with dilute samples that have large volume and/or low nucleic acid content. These devices can be manually operated using pipettors, can be adapted to a gravity-driven system, or can be automated with pumps. The cards comprise (i) a body member having a plurality of external surfaces and fabricated to contain a continuous fluid pathway therethrough, the pathway comprising a first port, a second port, and a binding channel intermediate and in fluid communication with the first port and the second port, wherein the binding channel is open to one of the external surfaces of the body member; and (ii) a glass member affixed to the one of the external surfaces of the body member to provide a first unmodified flat glass substrate in fluid communication with the binding channel. The binding channel may be open to a second of the external surfaces of the body member, in which case the device further comprises a second glass member affixed to the second external surface of the body member to provide a second unmodified flat glass substrate in fluid communication with the binding channel. The binding channel and affixed glass member(s) together form a binding chamber. Cells and nucleic acid are captured on the glass substrate(s). Such devices are conveniently fabricated by lamination of alternating polymeric and adhesive layers according to known methods, and glass microscope slides or cover slips are used as the glass members. To reduce the number of layers the binding channel and ports can be molded into a central member that is joined to one or more glass outer walls using adhesive or compression. See, for example, Reed et al., U.S. Patent Application Publication No. 20090215125 A1. The design of such devices permits fluids, including both liquids and gasses, to be passed through the device from one port to another. In this way solutions can be pumped back and forth through the binding channel to increase washing and elution efficiency, and air can be pumped through between washes and after the final wash to remove residual buffer and dry the bound nucleic acid. Such devices can be configured in a variety of ways with respect to introduction and removal of reagents, such as by adding additional ports and by varying the position of access ports.

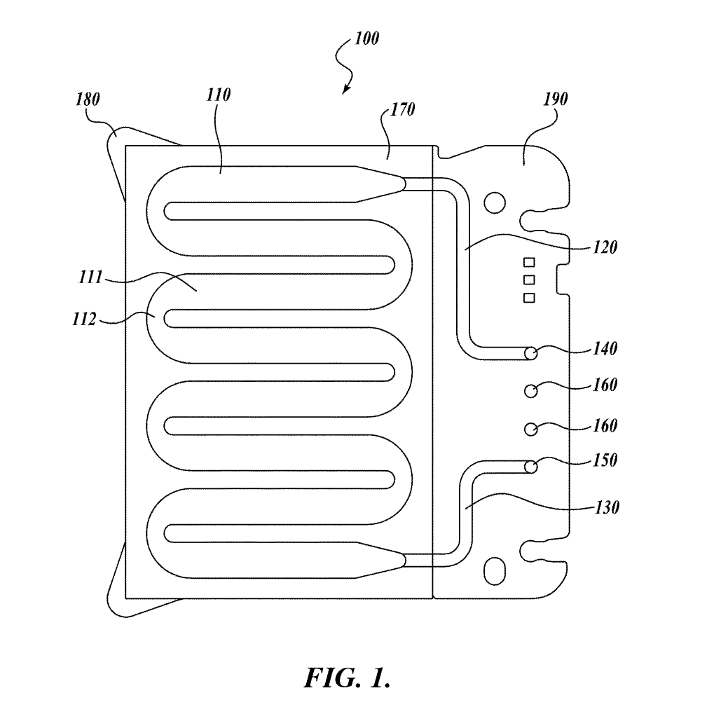

An example of an S-channel card for use within the present invention is illustrated in FIG. 1. Device 100 comprises an unmodified, flat glass substrate 170 for cell and nucleic acid binding and is also adapted for use with an optional manifold that can connect to a plurality of such devices. In the illustrated embodiment, flat glass substrate 170 is a 2×3 inch microscope slide, which, together with surface element 190, provides the illustrated external surface. Device 100 comprises S-shaped binding chamber 110, in which linear segments 111 are wider than bends 112. This device further comprises first and second channels 120 and 130, respectively, through which fluids are introduced into and removed from the device. First channel 120 is accessed via first port 140. Second channel 130 is accessed via second port 150. One or both of first port 140 and second port 150 may be equipped with a pipette interface (not shown) to receive and seal to a disposable pipette tip. A plurality of additional channels 160 pass through the device, which channels may be used to join the device to additional components. The illustrated device can be provided with an identification tag (not shown), such as a barcode, QR code, or RF tag, for sample tracking. The illustrated arrangement allows flow-through operation of the device, wherein liquids are introduced via one of the ports and withdrawn via the other of the ports. In the alternative, the device may be operated in a back-and-forth mode wherein reagents are introduced and withdrawn via one of the ports, and the other of the ports is used exclusively for withdrawal of the final sample so as to reduce the chance for contamination of eluted nucleic acid. Device 100 is constructed by laminating a plurality of individual elements into body member 180, to which glass substrate 170 is attached. Individual elements are joined using silicone adhesive. Material thicknesses and the number of layers can be varied to obtain different device thicknesses and volumes. In one embodiment, a glass substrate 170 is used on each of the front and back faces of the device. In this embodiment, both external layers of device 100 are formed by the combination of the glass substrates 170 with elements 190. Other devices of this type are disclosed by Reed et al., U.S. Patent Application Publication No. 20090215125 A1.

The invention provides for the isolation of cellular microorganisms, such as bacteria and yeasts. Of particular interest are those species that are pathogenic or are common contaminants in such areas as food processing, manufacturing, and the environment. One group of particular interest is the Gram-negative bacteria, such as E. coli.

Any sample containing microorganisms can be used. It has been found that bacteria in complex samples such as blood or culture media will bind to smooth glass surfaces. Common blocking agents such as albumin and mannose did not prevent binding. Various binding media can be used, provided that they are of low ionic strength and essentially free of cell precipitants. Known cell precipitants include alcohols, particularly aliphatic alcohols such as methanol, ethanol, isopropanol, and butanol; and polyethylene glycols or other high molecular weight polymers as disclosed by Rudi et al., U.S. Pat. No. 6,617,105 B1. Effective concentrations of some cell precipitants can be toxic to microorganisms. Within certain embodiments of the invention, the binding solution is free of alcohol and/or other cell precipitants. Particularly useful media include water, culture media, or various buffered formulations. Suitable binding medium formulations can include buffers such as Tris-Cl, citric acid, or HEPES at any concentration and at pH values above 7.0. Salts such as NaCl, KCl, sodium acetate or others may be added so long as a low ionic strength is maintained. Binding solutions may also contain detergent, such as polysorbate 20 (TWEEN-20) or 4-(1,1,3,3-Tetramethylbutyl)phenyl-polyethylene glycol (TRITON X-100) at a concentration less than 10% or SDS at a concentration less than 0.25%. A typical such binding medium is 10 mM Tris-Cl pH 8.0, 1 mM EDTA. Any volume of binding medium may be used, or no additional binding medium may be used. In the latter case, a sample containing microorganisms is loaded directly into the extraction device without any addition of reagents. The binding medium may be useful as a means to dilute substances that potentially may interfere with binding or microorganisms to the glass substrate.

The use of low ionic strength media that are essentially free of cell precipitants maintains microorganism viability through the isolation process, thereby allowing the isolated microorganisms to be cultured, including culturing within the extraction chamber. This feature of the invention facilitates the analysis of very dilute samples where the isolation of sufficient numbers of microorganisms for further analysis is otherwise challenging. This feature also allows measurement of relative numbers of viable and non-viable microorganisms by assaying for pre-rRNA (Cangelosi and Brabant, J. Bact. 179:4457-4463, 1997).

The methods disclosed herein allow relatively large volumes of microorganism suspensions to be exposed to glass and the bound microorganisms or extracted nucleic acid to be resuspended in a relatively small solution volume, thereby concentrating the microorganisms or nucleic acid. Thus, the methods are well suited to detection and isolation of low levels of microorganisms and allow the concentration of dilute cell suspensions without the need for centrifugation or filtration. Examples of such dilute solutions include water samples that may contain low levels of cells, and waste streams from food processing or other industrial processes that may exhibit very low levels of microbial contamination. In this aspect of the invention, volumes of solution hundreds of times the volume of the extraction device binding chamber can be processed through the device. While these large samples may be divided into aliquots and processed in a batch-wise fashion with each aliquot allowed to stand in the device for the desired contact time, it is much more convenient to utilize a flow-through process calibrated to provide the desired contact time between the cells in the solution and the glass substrate(s) of the binding chamber. Thus, sample volumes can be increased to two, four, eight, ten or more multiples of the binding chamber volume. In certain embodiments of the invention, the sample volume that is introduced into the extraction device is up to 30 times, 40 times, or 50 times the volume of the binding chamber. In other embodiments, volumes up to 100 times or 200 times the volume of the binding chamber are processed through the extraction device. Larger volumes can be processed, with time being the practical limiting factor. For water testing, sample volumes of 75-300 mL are particularly advantageous. For flow-through operation, the sample is passed through the binding chamber in a continuous flow mode at a flow rate selected to provide the desired contact time. Flow rates from 10 μL/minute to 10 mL/minute are practical for glass-walled devices. For example, when using a 1.5-mL extraction device, a flow rate of 50 μL/minute provides a effective contact time of 30 minutes.

For demonstration purposes, several colonies from a freshly grown plate of E. coli were scraped into 1 ml of growth medium. A sample of this bacterial suspension was mixed with 200 μl of pure water and loaded into a vessel comprising a serpentine binding channel (Reed et al., U.S. Patent Application Publication No. 20090215125 A1) (also known as an “S-channel card”). The suspension was allowed to sit for at least 10 minutes at room temperature to allow bacteria to adsorb to the glass. The liquid was removed, and the channel was rinsed at least four times with pure water. Any wash buffer formulation that does not lead to bacterial lysis can be used in place of water. Suitable alternative wash buffers include buffers such as Tris-Cl, citric acid, or HEPES at any concentration and at pH values above 7.0. Salts such as NaCl, KCl, sodium acetate or others may be added, as well as carrier components such as serum albumin at concentrations less than 10%. The remaining drops of wash medium are removed as completely as possible using a pipetor. The methods of the invention have also been used to capture Aeromonas hydrophila cells on a glass substrate.

While not wishing to be bound by theory, bacteria are believed to settle on glass and bind by ionic and non-ionic forces involving cell-wall interactions between proteins and/or carbohydrates on the cell and silanol or silicate functional groups on the glass substrate(s). Bound bacteria can be detected by a variety of methods known in the art, some of which are disclosed in more detail below.

Microorganisms can be isolated from samples of simpler composition such as water, or more complex samples such as blood or platelet concentrates. Blood samples contain large numbers of both red and white cells. When blood was used as a sample, it was clear that at least the red cells did not adhere to the glass. This means that by simply moving a blood sample over a smooth glass surface, bacteria can easily be separated from blood. The invention is therefore useful in applications such as sepsis testing where any bacterial contamination of blood is a problem. Additional applications of the invention are described in more detail below. For purposes of brevity and illustration, these examples describe processes comprising isolation of bacteria, but can be applied to other microorganisms such as yeasts. Additional applications will be evident to those of ordinary skill in the art. Illustrative applications are disclosed below.

(1) Concentration of bacteria for analysis by DNA Probes. Concentration of bacteria on glass substrates can be used to concentrate dilute bacterial samples. Lysis reagents are added to liberate DNA, which is then bound directly to the glass substrate (e.g., within an S-channel card). After a washing step, the bound nucleic acid can then be either eluted for off-card analysis, or amplified directly on the card (and detected thereon for a true all-in-one, lab-on-a-chip analysis). Details of DNA extraction and downstream analysis are disclosed by Reed et al., U.S. Patent Application Publication No. 20090215125 A1.

(2) Concentrate and Culture Bound Bacteria. It is known that bacteria bound to some surfaces will maintain the ability to divide and grow. However, concentrating bacteria from dilute suspensions can result in loss of cells and potentially false positive readings. As disclosed above, the methods of the invention allow larger volume, dilute samples to be run over unmodified, smooth glass substrates whereby the bacteria adhere to the glass. Growth medium is then loaded onto the bound bacteria allowing them to divide, much as would happen on a Petri dish. Newly divided cells can be released to the surrounding medium and then removed, or can be allowed to bind back down onto the glass substrate. Viability of bound cells was demonstrated by first starving the bound cells by incubating them in water, then incubating them in culture medium and assaying for the presence of pre-rRNA, which is indicative of growing cells (Cangelosi and Brabant, J. Bact. 179:4457-4463, 1997). This property could be an advantage for the following reasons: (i) to increase detection levels for further analysis within the extraction device; (ii) to collect bacterial flow-through (with expanded amounts of bacteria) for further microbiological work; and (iii) viability testing.

(3) Bacterial Detection by Staining. A variety of stains are currently used to visualize bacteria on glass microscope slides. Because the sensitivity of staining methods is limited, it can be an advantage to concentrate bacteria in samples by centrifugation, but this can lead to loss of bacteria in samples and lower sensitivity. Concentrating bacterial samples on smooth glass provides a simple alternative to centrifugation. A silicone (or other material) gasket comprising a void in the shape of an S-channel can be sandwiched between two glass slides and held in place with clamps. The sample can then be run into the sandwiched channel, held to allow bacterial binding to the glass, flushed through with additional sample run through, and repeated as necessary. One slide of the sandwich can be coated to discourage binding and allow binding only to one slide. The gasket can then be removed and the slide(s) stained as needed. This process can be used, for example, in Mycobacterium tuberculosis testing that otherwise requires sample concentration by centrifugation.

(4) Immunoassay Applications. The procedure disclosed above can be applied to concentrate bacteria from dilute clinical samples. The use of glass tubes or other glass-walled devices in analyzing dilute clinical samples can be a great advantage. In this application, the sample is run directly through a short glass tube. Cycles of sample addition, waiting for binding, and removal of sample are then performed until done. In the alternative, the sample solution can be allowed to slowly flow through the device. Immunoassay reagents are then run through the device, and bound bacteria detected colorimetrically or by more sensitive chemiluminescence. This type of system can be adapted for automated operation as disclosed in more detail below.

Bound microorganisms can be lysed directly on the glass. Nucleic acids released after lysis bind directly onto the glass and can be subsequently recovered. To lyse bound bacteria, a lysis mix is prepared. A typical mix combines one-third volume of a lysis buffer, one-third volume of water, and one-third volume of pure ethanol. To aid in lysis and facilitate capture of nucleic acids, the lysis buffer may contain a chaotrope such as guanidine thiocyanate, guanidine hydrochloride, or other known chaotrope at a minimum concentration of 1M. The lysis buffer also contains detergents such as Tween-20 or Triton X-100, both at a minimum concentration of 0.5%, sodium dodecyl sulfate at a minimum concentration of 0.05%, or N-lauryl sarcosine at a minimum concentration of 0.05%. One such useful formulation contains 4M Guanidine thiocyanate, 50 mM Citrate pH 6.0, 20 mM EDTA, 10% Tween-20, 3% Triton X-100. A protease may be added to help induce bacterial lysis, although with some bacterial species, a protease may not be necessary. Suitable proteases include Proteinase K, Subtilisin, and Pronase, each added to a minimum concentration of 10 μg/ml. In one method, proteinase K is added to a final concentration of 400 μg/ml. This lysis mixture is passed into the channel and allowed to sit for at least 5 minutes at room temperature. Some protease activity may be enhanced by incubation at higher temperatures, so that the incubation may be accomplished at temperatures up to 55° C. The lysis mixture is then removed. The channel is washed a minimum of one time, but preferably at least 2 times with a first wash buffer that contains a chaotrope such as guanidine thiocyanate or guanidine hydrochloride at a minimum concentration of 1M. One such suitable formulation contains 2M Guanidine thiocyanate, 30 mM Citrate pH 6.0, 13 mM EDTA, 33% pure ethanol. The channel is then washed as few as two times but preferably six times with a second wash buffer (e.g., 70% ethanol in 20 mM Tris-Cl pH 7.0). The formulation of the second wash buffer is less important as long as it contains at least 70% ethanol. The ethanol maintains binding of nucleic acid to glass in the absence of chaotrope. The last drops of wash buffer are removed, and the channel is fully dried under vacuum. Formulation of lysis buffers for other microorganisms is within the level of ordinary skill in the art.

Bound nucleic acid can be eluted from the glass substrate by sweeping a small volume of low ionic strength buffer such as TE (10 mM Tris-Cl pH 8.0, 1 mM EDTA) over the surface of the glass. Any very low ionic strength buffer can be used to elute bound nucleic acid at pH values of at least 5.0 or greater. These elution buffers contain very low concentrations of buffer component such as 50 mM or less, EDTA as a preservative, and no additional salt. Alternatively, water may be used to elute bound nucleic acid, although it may be necessary to carefully monitor the pH of the water. Very acidic buffers may not elute bound nucleic acid efficiently.

Not all of the liberated nucleic acid released by the lysis mixture may be bound by the glass substrate. Therefore the lysis mixture that is removed from the channel after the lysis step may contain significant amounts of nucleic acid. In this case, the lysis mixture may be applied to a fresh glass-walled extraction device for purification, or may be purified directly using another method, such as silica particle-based purification.

Liberated nucleic acids commonly contain both RNA and DNA. These can be detected by commonly available methods such as PCR, NASBA, or other amplification techniques or detection technologies. In addition to NASBA, useful isothermal amplification techniques include branched DNA (Alter et al., J. Viral Hepat. 2:121-132, 1995; Erice et al., J. Clin. Microbiol. 38:2837-2845, 2000), transcription mediated amplification (Hill, Expert. Rev. Mol. Diagn. 1:445-455, 2001), strand displacement amplification (Walker, PCR Methods and Applications 3:1-6, 1993; Spargo et al., Mol. Cell. Probes 10:247-256, 1996), helicase-dependent amplification (Vincent et al., EMBO Rep. 5:795-800, 2004), loop-mediated isothermal amplification (Notomi et al., Nucl. Acids Res. 28:E63, 2000), INVADER assay (Olivier et al., Nucl. Acids Res. 30:e53, 2002; Ledford et al., J. Mol. Diagn. 2:97-104, 2000), cycling probe technology (Duck et al., BioTechniques 9:142-148, 1990; Cloney et al., Mol. Cell. Probes 13:191-197, 1999), rolling circle amplification (Fire and Xu, Proc. Nat. Acad. Sci. USA 92:4641-4645, 1995; Liu et al., J. Am. Chem. Soc. 118:1587-1594, 1996), and Q-beta replicase (Shah et al., J. Clin. Microbiol. 32:2718-2724, 1994; Shah et al., J. Clin. Microbiol. 33:1435-1441, 1995). For a review of isothermal amplification methods, see Gill and Ghaemi, Nucleosides Nucleotides Nucleic Acids 27:224-243, 2008. If amplification is to be carried out within the binding chamber, it may be beneficial to passivate a portion of the chamber, such as by silanization, to improve amplification performance. See, for example, Shoffner et al., Nucl. Acids Res. 24(2):375-379, 1996.

Quantitation of extracted nucleic acids is facilitated by the inclusion of a fluorescent compound within the elution buffer, thereby providing a rapid quality check on the extraction process while the extracted nucleic acids are still within the device. Thus, within one embodiment of the invention the nucleic acids are contacted with a fluorescent compound having a fluorescence intensity dependent on the concentration of nucleic acids, and the fluorescence of the fluorescent compound is measured. Representative fluorescent compounds include fluorogenic minor groove binder agents such as bis-benzimide compounds and intercalating fluorogenic agents such as ethidium bromide, and commercially available fluorescent dyes (e.g., SYBR Green; Invitrogen Corp.). A preferred dye is a bis-benzimidine (BB) dye disclosed by Reed et al., U.S. Patent Application Publication No. 20060166223 A1, which gives a strong fluorescent signal (detection at 460 nm, 40 nm filter slit width) when excited at 360 nm (40 nm slit width) and is compatible with amplification procedures. The BB dye is selective for dsDNA but can also detect RNA. A popular green fluorescent dye, SYBR green (Invitrogen Corp.) is often used in so called “real time” PCR or quantitative PCR. Much like the BB dye, SYBR green can be used to both quantitate the extracted DNA before amplification and monitor the gene-specific increase during PCR. The use of fluorogenic DNA dyes or DNA probes in isothermal nucleic acid tests such as NASBA is also known.

Extracted nucleic acid can be stored on the unmodified, smooth glass substrate for extended periods without the need for refrigeration or freezing. Storage is facilitated by the use of enclosed vessels, such as the S-channel cards disclosed above. DNA has been found to be particularly stable when stored in this manner.

Glass-walled extraction devices are well suited to automated operation, and a plurality of devices can be operated in parallel using an appropriate manifold. In a representative arrangement, liquid flow is computer-controlled using an automated system built with commercially available components. A plastic (e.g., polycarbonate or acetal) manifold is used to connect laminated, flat-glass devices (“cards”) and the automated system using flexible tubing. To make a fluidic connection with the cards, o-rings in the manifold seal against the flat surface of the card, around the port openings, by compression provided by screw closures, clamping jigs, spring pressure, or other suitable means. In this system, samples are prepared and loaded into the cards manually. Cards are then connected to the pump, and the reagents are flowed through the cards automatically. Reagent selection can be accomplished using a custom manifold and solenoid valves or by using a rotary valve with multiple inputs and a common output. Reagent movement is accomplished using a four-channel peristaltic pump (e.g., Ismatec, Glattbrugg, Switzerland). A flow splitter between the valve and pump allows four cards to be processed at once. An on-board micro controller operates the solenoid valves and communicates with the pump via serial connection. Custom software on a serially connected computer allows the user to control the system. The above-disclosed arrangement can be adapted to other extraction devices, including tubular devices, and can further provide for automated nucleic acid extraction from captured microorganisms using additional reagent cycles (lysis, wash, elution). Extraction channels can be dried using a low pressure air stream (˜1.5 L/min), and wastes pumped into a 1-L bottle with a vent hole.

The S-channel and tubular devices disclosed by Reed et al. (ibid.) can be adapted to utilize gravity to drive fluid flow. The device is arrayed with the binding chamber in a substantially horizontal orientation and the sample solution is introduced into the binding chamber. One of the first and second ports is then selected as an outlet and the other as an inlet for the remaining reagents. The outlet is connected to a siphon tube via a first end of the tube and to a vented outflow receptacle via a second end of the tube, whereby the siphon tube provides fluid communication between the outlet and the outflow receptacle. A portion of the siphon tube is disposed at an elevation above the binding chamber and the second end is not above the first end. A buffer reservoir is then connected to the inlet at an elevation above the binding chamber such that gravity forces buffer to flow through the channel displacing the sample solution through the siphon tube into the receptacle. The buffer reservoir is refilled and the process repeated as necessary to move all reagents through the binding chamber. Gas bubbles may be introduced into the chamber between reagents to reduce mixing.

The invention is further illustrated by the following, non-limiting examples.

### EXAMPLES

### Example 1

Capture of bacteria on unmodified, smooth glass was compared to capture on magnetic beads with immobilized antibodies. Experiments were carried out in S-channel extraction devices (Reed et al., US 20090215125 A1) at room temperature.

Biotinylated polyclonal antibody against E. coli (at a concentration of 4 mg/ml) was obtained from Abcam Inc. (Cambridge, Mass.). Streptavidin-coated magnetic beads (at a concentration of 1.9 g/ml) were obtained from Bangs Laboratories (Fishers, Ind.). Prior to use in S-channels, beads were saturated with biotinylated antibody. Based on the binding capacity of the beads and the concentration of the antibody, it was estimated that 3 μl of antibody would saturate 10 μl of beads. Therefore 4 μl of antibody (excess above saturation) was added to 10 μl of beads and allowed to adsorb for 10 minutes. A binding buffer (100 μl TE) was added to the beads and mixed well.

Aliquots of beads with or without bound antibody, thoroughly suspended in binding buffer, were then transferred to S-channels using a pipetor. Using a magnet, the beads were held in place and rinsed twice with binding buffer to remove excess antibody. A suspension of E. coli cells in 200 μl of binding buffer was then added to the channel. Using the magnet, the particles were mixed into the suspension of cells. The S-channel cards were incubated for 30 minutes to allow bacterial capture, then the bacterial suspension was removed from the channel. Then, using a magnet to hold the particles in place, the particles were washed 3 times with binding buffer to remove unbound cells. Then the beads were covered with binding buffer. The magnet was used to suspend the particles in the buffer. After sitting for a minute, the magnet was used to hold the beads in place while as much buffer as possible was removed from the channel with a pipetor. The channel at this point had the particles and drops of binding buffer.

A lysis buffer was prepared by mixing 0.4 ml lysis buffer concentrate (4M Guanidine thiocyanate, 50 mM Citrate, pH 6.0, and 20 mM EDTA, 1% Triton X-100, 10% Tween-20) with 0.4 ml water, 0.04 ml Proteinase K (at 10 mg/ml), and 0.4 ml ethanol. The beads in the channel were covered with the lysis mix while being held in place by the magnet. The magnet was then used to suspend the particles in the lysis mix. The channels were incubated at room temperature for 30 minutes. The lysis mix including the beads was then removed from the channel. The channels were washed twice with a buffer consisting of 2M Guanidine HCl, 33 mM Citric Acid pH 6.0, 13 mM EDTA, 33% ethanol (wash 1). The channels were then washed six times with a buffer consisting of 20 mM Tris pH 7.0, 70% ethanol (wash 2), and finally dried under vacuum for 30 minutes.

In some cases, the ability of glass to bind bacteria in blood was tested. In these cases, bacteria were suspended directly into a sample of blood. The blood sample was loaded directly onto the channel containing the magnetic beads. All other steps were performed as described.

Samples containing bacteria and antibody only were prepared essentially as described above. Bacteria and antibody were combined in TE and added to S-channels as described. Binding, washing, and lysis were carried out as described, but no magnet was used.

To elute bound nucleic acids, 75 μl of TE buffer was swept over the channel with a pipetor and removed from the channel.

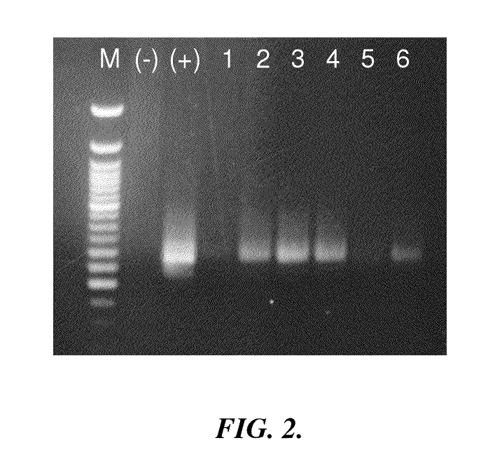

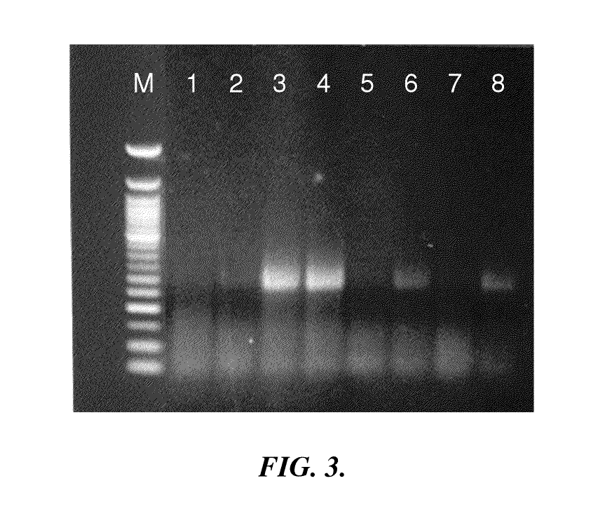

Eluted nucleic acid was amplified by NASBA using E. coli pre-rRNA primers EcAmp001 (SEQ ID NO:1) (AATTTAATACGACTCACTATAGGGAAGTAGTTATCCCCCTCCATC) and Ec005 (SEQ ID NO:2) (GATACGGATTCTTAACGTCGC). Results are shown in FIGS. 2 and 3. As shown in FIG. 2, the complete capture mix gave the same results as other combinations. This result indicates that bacterial binding was most likely to the glass only, suggesting that the bacteria bound preferentially to smooth glass over the antibody-coated beads. The experiment also demonstrated that bacteria could be efficiently captured from blood and could be detected by amplification. As shown in FIG. 3, bacteria bound to unmodified, smooth glass without beads or antibody, and were captured from blood in the S-channel device.

### Example 2

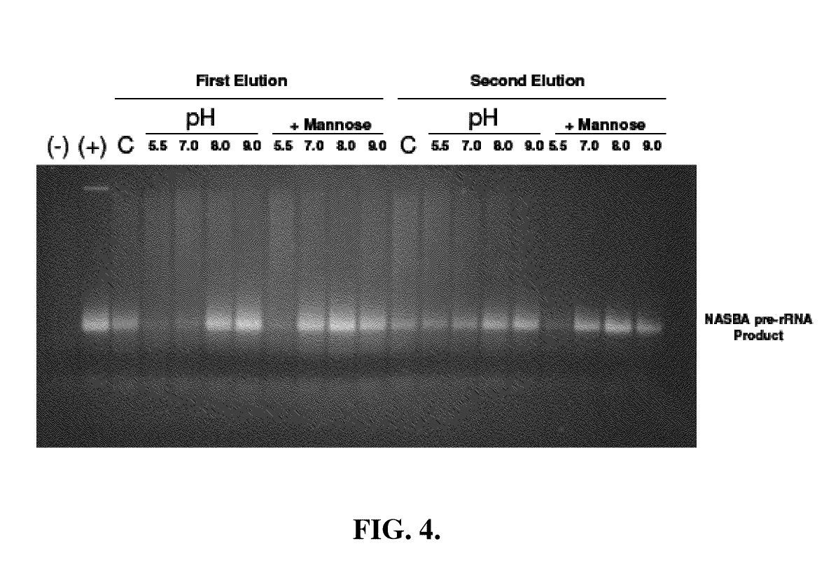

An assay was conducted to demonstrate binding of bacteria to naked glass. A few colonies of bacteria (E. coli) were scraped off a plate into growth medium (brain heart infusion) and placed at 37° C. to revive the bacteria. Twenty μl of cell suspension was diluted into 100 μl or 200 μl of water (control) or binding buffer (0.1M Tris with 1% BSA adjusted to the various pHs shown in FIG. 4). Mannose was included in some samples at a concentration of 0.5 mg/ml. The bacterial suspensions were loaded onto S-channel DNA extraction devices and allowed to sit for about 15 minutes. The bacterial suspensions were then removed. Unbound bacteria were washed out of the devices by filling the first rung of the S-channel with 200 μl binding buffer. The buffer was allowed to sit 1-2 minutes and then removed. The wash was repeated three additional times. A lysis buffer was then prepared as disclosed in Example 1 with 200 μg of Proteinase K. 100 μl of this buffer was loaded into each device to cover the area where bacteria were bound. The lysis mix was incubated at room temperature for 30 minutes. During this incubation, bacteria were lysed and released nucleic acids were bound to the glass simultaneously. The lysate was removed, and the devices were washed 3 times with wash 1 and 6 times with wash 2 (Example 1). The devices were then dried under vacuum. Bound nucleic acids were eluted with two 75-μ1 aliquots of TE. The 16s pre-rRNA was amplified using NASBA with primers specific for E. coli pre-rRNA as in Example 1 (SEQ ID NO:1 and SEQ ID NO:2). Products of the amplification were displayed on a 2% agarose gel. The results of one analysis are shown in FIG. 4.

This experiment investigated whether or not bacterial binding to glass could be inhibited by varying the pH or by the addition of the sugar mannose. Mannose has been reported to inhibit binding of bacteria to silicate surfaces (Buss, H. L., Brantley, S. L., and Liermann, L. J. (2003) Geomicrobiology J. 20: 25-42). However, as shown in FIG. 4 this inhibition was not observed. At pHs below 8.0, it appeared that binding of bacteria was lowered, although when mannose was included, it appeared that some binding at pH 7.0 was restored. The pattern for the second elutions was similar but not identical. This difference was probably due to influences such as inhibitors or carryover reagents that may influence the first elution patterns but were removed by the first elution.

Note that the control and the pH steps all had approximately the same number of bacteria, but the signals look stronger at higher pH. These results suggest that pH enhances bacterial binding or that pH somehow affects viability or stability of pre-rRNA.

Lysis and nucleic acid binding occur concomitantly in this experiment. Thus, an amplification (e.g., NASBA) mix could be loaded into the channels and bound nucleic acid amplified. Capture of bacteria coupled with nucleic acid extraction and pre-rRNA amplification as demonstrated here exploits and extends the full capability of the extraction device. The channel design should allow flow-through concentration of bacteria from water by binding nonspecifically to the glass.

### Example 3

Bacterial binding to glass pipettes was demonstrated. Two variations of the experiment were conducted, the first with increasing volumes of E. coli suspension, and the second a series of dilutions of the suspension. Standard long Pasteur pipettes were used throughout.

Several runs of colonies were scraped from a fresh plate of E. coli and suspended in 8 ml TE. Lots of bacteria were used in order to isolate large enough amounts of nucleic acid as to be detected by fluorescence. The suspension was quite cloudy with bacteria at this point. The entire experiment was done with this suspension. All points were done in triplicate.

Procedure: (1) Draw up a bacterial sample in the pipette until it is just above the neck (where the diameter increases). Seal the top of the pipette with tape and hold vertically for 30 minutes. (2) Remove the tape to allow the suspension to run out of the pipette. Wash the pipette three times with 1 ml TE to remove unbound bacteria. Remove as much wash as possible. (3) Draw up 0.4 ml of lysis buffer (Example 1) into the pipette. Lay the pipettes on their sides and roll them back and forth periodically for 40 minutes so that the pipette inner surfaces are bathed in lysis buffer. This step causes bacterial lysis and binding of liberated nucleic acid. (4) Pipettes are then placed in a plastic manifold to hold them vertically and washed 2× with wash 1 (Example 1), and 5 times with wash 2 (Example 1). (5) The pipettes are dried under vacuum. (6) To elute bound nucleic acid, 100 μl of TE is drawn into the barrel of the pipette, and the pipette is rotated to completely coat the interior surface, and then placed vertically into a tube to drain. This is repeated twice more for a total of 3 elutions. (7) Samples (20 μl) are quantitated with both PICOGREEN fluorescent dye (Invitrogen Corporation) (strongly detects DNA only), and RIBOGREEN fluorescent dye (Invitrogen Corporation) (which detects RNA and DNA).

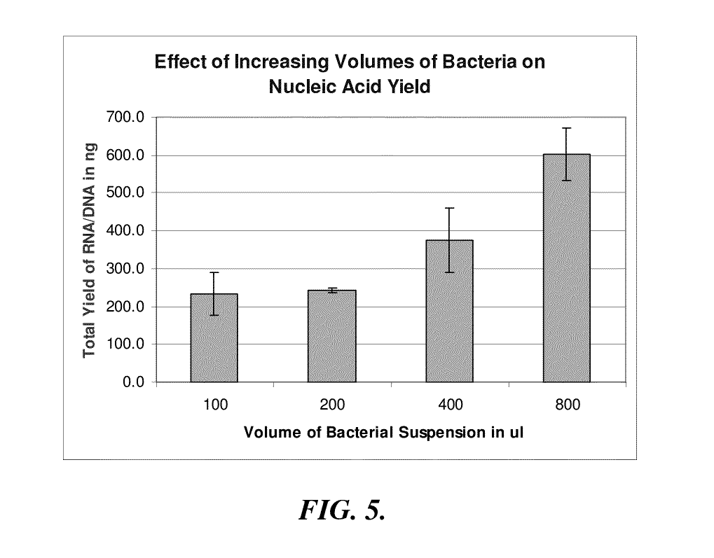

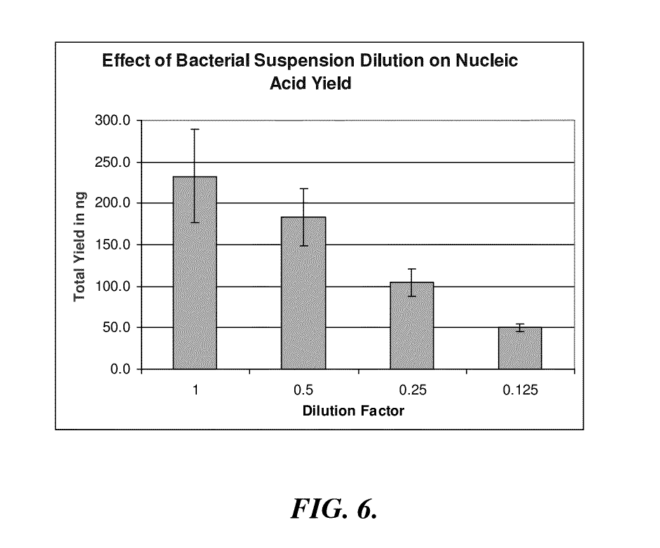

The results shown in FIGS. 5 and 6 are from the RIBOGREEN quantitation, as this reagent probably reflects better total recovered nucleic acid. For the first data set, increasing quantities of bacterial suspension were drawn into the pipet. Increasing amounts of suspension should cover more glass surface area, resulting in the binding of increasing amounts of bacteria. As shown in FIG. 5, nucleic acid yields increased with increased volumes of bacteria (increased surface area gave increased nucleic acid level), but the effect was not quantitative. Yields may have been limited by the somewhat inexact procedures used in this set of experiments.

In the second variation a sample of the suspension was taken, and two-fold dilutions were prepared. See FIG. 6. Again, yields were not directly proportional to the dilution factor.

### Example 4

A suspension of E. coli cells was prepared by scraping a portion of a colony from an agar plate into 10 mL of water. S-channel cards were filled with the bacterial suspension and allowed to sit for 30 minutes to allow binding of cells. The cell suspension was then removed, and unbound cells were removed by filling the channels with water, removing the water, and then repeating for a total of two washes. Bacteria incubated in water are depleted in markers of cell viability, in this case pre-rRNA. To demonstrate that the cells remained viable when bound to glass, channels were first filled with water and incubated at 37° C. for 1 hour to deplete pre-RNA. The water was then removed from all channels. Then, some channels were filled with trypticase soy broth (TSB), a common rich growth medium for bacterial cells. Channels filled with TSB were incubated for 1 hour at 37° C. for 1 hour to allow expression of the viability marker pre-rRNA. Bound cells were lysed in the card, and nucleic acid was recovered and amplified as described in example 1.

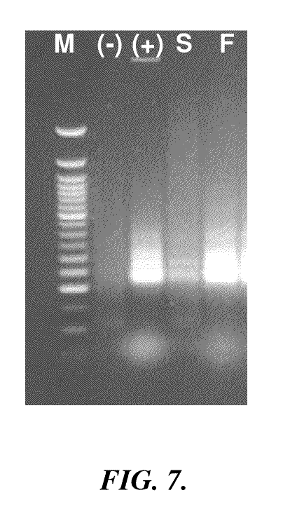

Results are shown in FIG. 7. Cells maintained in water lost their pre-rRNA and yielded a relatively dull NASBA band on the agarose gel of the amplification products. In contrast, cells incubated in TSB broth yielded a much brighter band on the gel. This result demonstrated that the cells retained viability when bound to the glass surfaces.

A second experiment was conducted to observe the effects of antibiotics on bound cells. S-channel cards with bound E. coli cells were prepared as in the viability experiment above. Bound cells were incubated in water at 37° C. for 1 hour. The water was removed and replaced with TSB broth containing Ampicillin or Tetracycline at 50 μg/ml. Bacteria were incubated for 1 hour at 37° C. In the case of antibiotic treatment, antibiotic-containing broth was removed and replaced by broth without antibiotic in order to assess whether the cells remained viable after antibiotic treatment. Bacteria were then incubated for an additional hour at 37° C. to allow bacteria a chance to recover. Bound cells were lysed in the card, and nucleic acid was recovered and amplified as described in example 1.

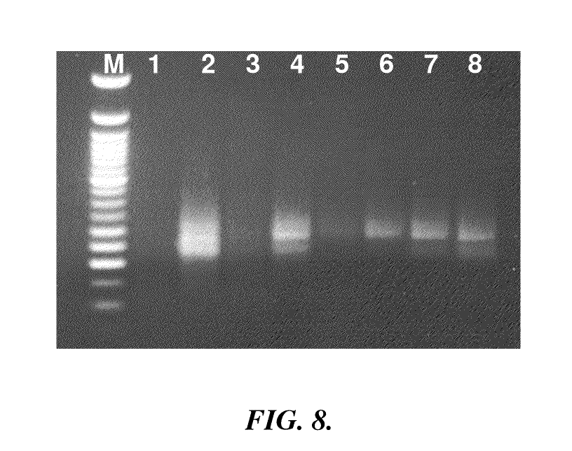

Results are shown in FIG. 8. Ampicillin is known to be bacteriocidal, or has the ability to kill the bacterial cells by altering their cell wall. As expected, cells treated with ampicillin could not be revived when transferred into TSB. Ampicillin-treated cells yielded a relatively dull fluorescent agarose gel band relative to cells not treated with ampicillin. In contrast, tetracycline is a bacteriostatic compound; treated cells are not killed but become quiescent and unable to grow. When the tetracycline is removed, cells are revived and can divide as before treatment. As predicted, tetracycline-treated cells yielded the same amount of fluorescence as the untreated cells. These results demonstrated that both viability and gene expression can be assessed with glass-bound bacteria.

### Example 5

In another experiment, bacterial capture on glass was performed in the presence of various detergents. A suspension of E. coli cells was diluted in a binding buffer consisting of TE with 1% bovine serum albumin (BSA), and the same TE with 1% BSA with the addition of 1% Tween-20, 1% Triton X-100, 1% Tween-20 together with 1% Triton X-100, or 0.1% sodium dodecyl sulfate (SDS). Cell suspensions were applied to the S-channel cards in each of these buffers and allowed to sit for 20 minutes for binding. Channels were washed with the same binding buffer. Guanidine thiocyanate lysis buffer was added as described in Example 1 to simultaneously break open cells and bind liberated nucleic acid. Cards were washed and dried as in Example 1. Eluates from the cards were amplified with the NASBA reaction as in Example 1.

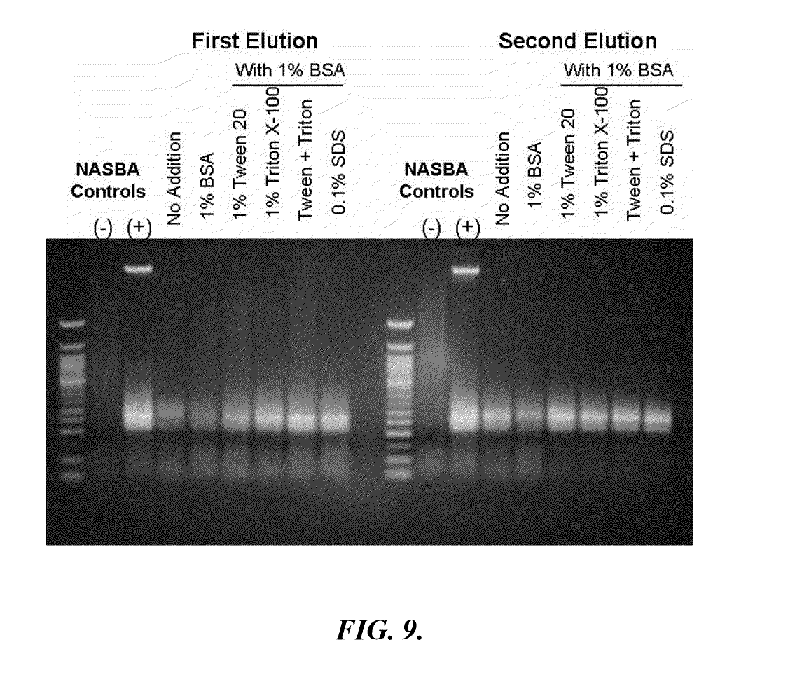

Results are shown in FIG. 9. 1% BSA seemed to lessen bacterial binding to glass by as much as 50% as evidenced by rather dull fluorescence on agarose gels of NASBA reaction products. However, Tween-20 partially restored binding in the presence of 1% BSA. 1% Triton X-100 increased binding further, but either the combination of detergents or 0.1% SDS yields were better (by greater than 50%) and showed little sign of inhibition of binding in the presence of SDS (agarose gel bands were brighter than the control bands, which had no additions). This experiment showed that bacterial capture can be influenced by a competitive binding substance (in this case BSA), but that certain detergent combinations can be used to restore and enhance high level capture of bacterial cells.

From the foregoing, it will be appreciated that, although specific embodiments of the invention have been described herein for purposes of illustration, various modifications may be made without deviating from the spirit and scope of the invention. Accordingly, the invention is not limited except as by the appended claims.

1. A process for isolating microorganisms comprising:
providing a device comprising an inner surface, an outer surface, a first port, and a second port, wherein the inner surface comprises an unmodified, smooth glass substrate and defines a binding chamber providing fluid communication between the first port and the second port;
contacting microorganisms in an aqueous solution with the unmodified, smooth glass substrate, wherein the solution is essentially free of cell precipitants; and
allowing the microorganisms to bind to the glass substrate to provide bound cells.

2. The process of claim 1 further comprising separating the aqueous solution from the bound microorganisms.

3. The process of claim 2 further comprising, following the separating step, adding a growth medium to the bound microorganisms and incubating the bound microorganisms, whereby the bound microorganisms increase in number.

4. The process of claim 1, further comprising lysing the bound microorganisms to produce a lysate.

5. The process of claim 4, further comprising:
adding a chaotropic salt to the lysate, whereby nucleic acid within the lysate binds to the unmodified, smooth glass surface; and
washing the bound nucleic acid to provide isolated nucleic acid.

6. The process of claim 5 further comprising amplifying the isolated nucleic acid.

7. The process of claim 6 wherein the amplifying step comprises isothermal amplification.

8. The process of claim 6 wherein the isolated nucleic acid is amplified within the binding chamber.

9. The process of claim 1 wherein the microorganisms are bacteria.

10. The process of claim 9 wherein the bacteria are Gram-negative bacteria.

11. The process of claim 1 wherein the microorganisms are yeast.

12. The process of claim 1 wherein the aqueous solution is essentially free of mineral salts.

13. The process of claim 1 wherein the aqueous solution comprises a detergent.

14. The process of claim 1 wherein the aqueous solution is a low-ionic-strength solution.

15. The process of claim 14 wherein the aqueous solution has an ionic strength less than 0.1 M.

16. The process of claim 1 wherein the aqueous solution is free of aliphatic alcohols.

17. The process of claim 1 wherein the aqueous solution comprises blood or a blood component.

18. The process of claim 1 wherein the unmodified, smooth glass substrate is flat.

19. The process of claim 18 wherein the binding chamber is a serpentine chamber of rectangular cross-section.

20. The process of claim 19 wherein the serpentine chamber is planar.

21. The process of claim 1 wherein the binding chamber encloses a volume x, and wherein a volume of the aqueous solution of at least 2× is contacted with the glass substrate.

22. The process of claim 21 wherein a volume of the aqueous solution of at least 10× is contacted with the glass substrate.
